(19) United States
(12) Patent Application Publication
(10) Pub. No.: US 2011/0306686 A1
(43) Pub. Date: Dec. 15, 2011

(54) ZEOLITE SUPPORTED RUTHENIUM CATALYSTS FOR THE CONVERSION OF SYNTHESIS GAS TO HYDROCARBONS, AND METHOD FOR PREPARATION AND METHOD OF USE THEREOF

(75) Inventors: Kandaswamy Jothimurugesan, Hercules, CA (US); Charles Leonard Kibby, Benicia, CA (US); Robert James Saxton, Pleasanton, CA (US)

(73) Assignee: Chevron U.S.A. Inc.

(21) Appl. No.: 12/797,439

(22) Filed: Jun. 9, 2010

(51) Int. Cl. B01J 29/068; C07C 1/12

(52) U.S. Cl. 518/715; 502/74

(57) ABSTRACT

A method for forming a catalyst for synthesis gas conversion and a synthesis gas conversion process impregnating a zeolite support wherein the catalyst contains ruthenium on a zeolite support, such as ZSM-5, ZSM-12, SSZ-32 or beta zeolite, and the product stream has less than 1 weight % C₂₁₊.

### TECHNICAL FIELD

The present disclosure relates to a process for the conversion of synthesis gas to liquid hydrocarbons in the presence of a zeolite supported ruthenium (Ru) catalyst containing an acidic functionality, the preparation of such catalyst, and the catalyst per se. More particularly, the present disclosure relates to conversion of synthesis gas to liquid hydrocarbons using a catalyst comprising ruthenium on a zeolite support that has been subjected to an activation treatment to provide improved activity and selectivity.

### BACKGROUND

High quality fuels remain in increasing demand with respect to the crude oil crisis and environmental impact. Fischer-Tropsch synthesis, which involves the production of hydrocarbons by the catalyzed reaction of carbon monoxide (CO) and hydrogen (H₂), can convert natural gas derived synthesis gas to liquid fuels and high-value chemicals. Fischer-Tropsch synthesis is one of the more attractive direct and environmentally acceptable paths to high quality transportation fuels.

Fischer-Tropsch catalysts are typically based on group VIII metals such as, for example, Fe, Co, Ni and Ru. The product distribution over such catalysts is non-selective and is generally governed by the Anderson-Schulz-Flory (ASF) polymerization kinetics.

What is needed is a Fischer-Tropsch catalyst that limits wax production in the Fischer-Tropsch reaction, and a method for forming such a Fischer-Tropsch catalyst comprising ruthenium, with its low water-gas shift activity.

### SUMMARY

According to one embodiment, the invention relates to a method for forming a catalyst for synthesis gas conversion, comprising impregnating a zeolite extrudate with ruthenium using a solution comprising a ruthenium salt to provide an impregnated zeolite extrudate, and activating the impregnated zeolite extrudate by a reduction-oxidation-reduction cycle.

According to another embodiment, the invention relates to a hybrid Fischer-Tropsch catalyst comprising a zeolite support impregnated with ruthenium and wherein the support is selected from the group consisting of ZSM-5, ZSM-12, SSZ-32 and beta zeolite.

According to yet another embodiment, the invention relates to a method of performing a synthesis gas conversion reaction, the method comprising contacting a hybrid Fischer-Tropsch catalyst comprising a zeolite support impregnated with ruthenium and wherein the support is selected from the group consisting of ZSM-5, ZSM-12, SSZ-32 and beta zeolite with synthesis gas at a reaction temperature of 160° C. to 300° C., and H₂ to CO ratio of 1.5 to 2.0 a total pressure of 3 to 35 atmospheres, and an hourly space velocity 100 to 5000 v/v/hour.

### DETAILED DESCRIPTION

A method for forming a catalyst for synthesis gas conversion is described. The method comprises impregnating a zeolite extrudate using a solution comprising a ruthenium salt to provide an impregnated zeolite extrudate and activating the impregnated zeolite extrudate by a reduction-oxidation-reduction cycle (“ROR activation”). In an embodiment, the supported ruthenium catalyst was prepared by the method of aqueous impregnation and vacuum drying, followed by calcinations. Also provided is a hybrid Fischer-Tropsch catalyst comprising a zeolite extrudate impregnated with ruthenium.

Zeolite supported mono- and bimetallic catalysts having Fischer-Tropsch activity have been found to be capable of limiting product chain growth in the Fischer-Tropsch reaction beyond C₂₀. Ruthenium alone, usually known as a promoter for cobalt, is a Fischer-Tropsch active metal that provides surprisingly low C₁₋₄ products from conversion of natural gas derived synthesis gases because of its low water-gas shift activity.

Ruthenium/zeolite catalysts with high activities for synthesis gas conversion to hydrocarbon liquids have been prepared using commercially available, alumina bound zeolite extrudates, e.g., ZSM-5, ZSM-12, SSZ-32 or beta zeolite. With ruthenium nitrate based compounds such as ruthenium nitrosyl nitrate, metal loading in a single step impregnation is limited to about 6 to 7 weight % ruthenium, even about 0.5 to 5 weight % for these alumina bound zeolites. Multiple impregnations may be needed, with intervening drying and calcination treatments to disperse and decompose the metal salts. The total ruthenium content can be varied from 0.1 weight % to 15 weight %. Calcination in air produces materials with lower activities than those formed by direct reduction of the ruthenium nitrate based compound. However, direct reduction on a large scale is considered to be undesirable since it is very exothermic and it produces a pyrophoric catalyst that must then be passivated before it can be handled in air. A low temperature reduction-oxidation-reduction cycle, described below in further detail, may be preferable to a single reduction step for the activation of ruthenium/zeolite catalysts for synthesis gas conversion.

Methods of formation of zeolite extrudates are readily known to those of ordinary skill in the art. Wide variations in macroporosity are possible with such extrudates. For the present application, without wishing to be bound by theory, it is believed that as high a macroporosity as possible, consistent with high enough crush strength to enable operation in long reactor tubes, is advantageous in minimizing diffusion constraints on activity and selectivity. In extrudate formation, strength is produced in a calcination step at high temperature. The temperature is high enough to cause solid state reactions between ruthenium oxides and alumina or aluminosilicate portions of the material, to form very stable, essentially non-reducible phases such as spinels. Consequently, it is preferred that the metal be added after the extrudate has been formed and has already undergone calcination since preparing an extrudate using a previously impregnated Ru-zeolite powder and an aluminum binder would result in a catalyst of much lower activity.

As used herein, the phrase “hybrid Fischer-Tropsch catalyst” refers to a catalyst comprising a Fischer-Tropsch component as well as a component containing the appropriate functionality to convert in a single-stage the primary Fischer-Tropsch products into desired products (i.e., minimize the amount of heavier, undesirable products). Thus, the combination of a Fischer-Tropsch component displaying high selectivity to sort-chain α-olefins and oxygenates with zeolite(s) results in an enhanced gasoline selectivity and an increased concentration of high-octane branched and aromatic hydrocarbons by promoting oligomerization, cracking, isomerization, and aromatization reactions on the zeolite acid sites. Gasoline-range isoparaffins also can be produced in a single reactor using hybrid catalysts by combining a ruthenium-based Fischer-Tropsch component with an acidic or bifunctional zeolite component in a mixture of separate Fischer-Tropsch and zeolite particles. The branched nature of the carbon chain products make them beneficial for transportation fuels having low temperature pour, cloud or freeze points, i.e. less than about −20° C. Any waxy products formed on the ruthenium component are cracked/hydrocracked by the acidic zeolite component into mainly branched hydrocarbons with limited formation of aromatics, a less desirable component in reformulated gasolines due to environmental concerns. In particular, in a single-stage reaction, the presently disclosed process provides:
0-20 for example, 1-15 or 4-14, weight % CH₄;
0-30 for example, 5-30 or 6-16, weight % C₂-C₄;
50-95, for example, 65-90 or 70-90, weight % C₅₊; and
0-2 weight % C₂₁₊.

As used herein, the phrase “zeolite supported ruthenium catalyst” refers to a hybrid catalyst wherein the ruthenium metal is distributed as small crystallites upon the zeolite support. The ruthenium content of the zeolite supported ruthenium catalyst can depend on the alumina content of the zeolite. For example, for an alumina content of about 20 weight % to about 99 weight % based upon support weight, the catalyst can contain, for example, from about 1 to about 20 weight % ruthenium, even from about 1 to about 5 weight % ruthenium, based on total catalyst weight, at the lowest alumina content. At the highest alumina content the catalyst can contain, for example, from about 1 to about 20 weight % ruthenium, even from about 2 to about 10 weight % ruthenium, based on total catalyst weight.

It has been found that synthesis gas comprising hydrogen and carbon monoxide can be selectively converted under synthesis gas conversion conditions to liquid hydrocarbons with a catalyst prepared by subjecting a zeolite supported ruthenium catalyst to an ROR activation procedure comprising the steps, in sequence, of (A) reduction in hydrogen, (B) oxidation in an oxygen-containing gas, and (C) reduction in hydrogen, the activation procedure being conducted at a temperature below 500° C. It has been found that the activation procedure of the present disclosure provides zeolite supported ruthenium catalyst with improved reaction rates when the catalyst is prepared by impregnation of a zeolite support with ruthenium. While ruthenium is used as a promoter for cobalt, it was surprisingly that significant carbon numbers in the fuel range with low amounts of C₄— products and low amounts of C₂₀₊ products could be achieved when combined with a zeolite, e.g. a ZSM-5, ZSM-12, SSZ-32 or beta zeolite.

Optionally, Re, Rh, Pt, Pd, Ag, Au, Mn, Zn, Cd, Hg, Cu, Pr or other rare earth metals can be added as a promoter to improve the activity of the zeolite supported ruthenium catalyst. Higher loadings of Ru without a promoter favor gasoline range products. Rhenium (Re) is a promoter which favors diesel range products. As an example, for a catalyst containing about 3 weight % ruthenium, the amount of rhenium promoter can be from about 0.1 to about 1 weight %, for example, from about 0.05 to about 0.5 weight % based upon total catalyst weight. The amount of rhenium would accordingly be proportionately higher or lower for higher or lower ruthenium levels, respectively. Catalyst levels of about 3 weight % have been found to best for 80 weight % ZSM-5 and 20 weight % alumina. The amount of ruthenium can be increased as amount of alumina increases, up to about 6 weight % Ru.

Suitable catalysts have from 0.8 to 1.2 weight % Ru and a support selected from the group consisting of ZSM-5 or beta zeolite with from 0.0 to 0.7 weight % Re. A catalyst with 1.5 to 2.5 weight % Ru and a ZSM-5 support without Re produces more hydrocarbons in the gasoline range than diesel range while still having less than 1 weight % above C₂₁⁺.

A zeolite is a molecular sieve that contains silica in the tetrahedral framework positions. Examples include, but are not limited to, silica-only (silicates), silica-alumina (aluminosilicates), silica-boron (borosilicates), silica-germanium (germanosilicates), alumina-germanium, silica-gallium (gallosilicates) and silica-titania (titanosilicates), and mixtures thereof.

Molecular sieves, in turn, are crystalline materials that have regular passages (pores). If examined over several unit cells of the structure, the pores will form an axis based on the same units in the repeating crystalline structure. While the overall path of the pore will be aligned with the pore axis, within a unit cell, the pore may diverge from the axis, and it may expand in size (to form cages) or narrow. The axis of the pore is frequently parallel with one of the axes of the crystal. The narrowest position along a pore is the pore mouth. The pore size refers to the size of the pore mouth. The pore size is calculated by counting the number of tetrahedral positions that form the perimeter of the pore mouth. A pore that has 10 tetrahedral positions in its pore mouth is commonly called a 10-ring pore. Pores of relevance to catalysis in this application have pore sizes of 8 rings or greater. If a molecular sieve has only one type of relevant pore with an axis in the same orientation to the crystal structure, it is called 1-dimensional. Molecular sieves may have pores of different structures or may have pores with the same structure but oriented in more than one axis related to the crystal. In these cases, the dimensionality of the molecular sieve is determined by summing the number of relevant pores with the same structure but different axes with the number of relevant pores of different shape.

Exemplary zeolite supports of the present disclosure include those which are fairly acidic Bronstead acids having Si to Al ratios of about 10 to 100. Examples are SSZ-26, SSZ-33, SSZ-46, SSZ-53, SSZ-55, SSZ-57, SSZ-58, SSZ-59, SSZ-64, ZSM-5, ZSM-11, ZSM-12, MTT (e.g., SSZ-32, ZSM-23 and the like), H-Y, BEA (zeolite Beta), SSZ-60 and SSZ-70. Preferred supports are ZSM-5, Beta, and SSZ-26. These molecular sieves each contain silicon as the major tetrahedral element, have 8 to 12 ring pores, and are microporous molecular sieves, meaning having pore mouths of 20 rings or less.

The zeolite supports can have a BET surface area of between about 100 m²/g and about 300 m²/g, for example, about 180 m²/g. Micropore volume for 80% ZSM-5 should be between about 90 and 112 μL/g, with lower volumes implying some occlusion or loss of micropore structure. The zeolite supports can further have porosity of between about 30 and 80%, total intrusion volume of between about 0.25 and 0.60 cc/g, and crush strength of between about 1.25 and 5 lb/mm.

Initially, the zeolite support can be treated by oxidative calcination at a temperature in the range of from about 450° to about 900° C., for example, from about 600° to about 750° C. to remove water and any organics from the zeolite support.

The method employed to deposit the catalytic metals of the present disclosure onto the zeolite support can involve an impregnation technique using a solution containing soluble ruthenium salt and, if desired, a soluble promoter metal salt which will not have a poisonous effect on the catalyst e.g., for example, rhenium salt, in order to achieve the necessary metal loading and distribution required to provide a highly selective and active catalyst. Any suitable ruthenium salt, such as ruthenium nitrate, chloride, acetate or the like can be used. Aqueous solutions for the promoters can be used in very small amounts. Nonaqueous solutions can also be used.

Suitable nonaqueous solvents include, for example, ketones, such as acetone, butanone (methyl ethyl ketone); the lower alcohols, e.g., methanol, ethanol, propanol and the like; amides, such as dimethyl formamide; amines, such as butylamine; ethers, such as diethylether and tetrahydrofuran; hydrocarbons, such as pentane and hexane; and mixtures of the foregoing solvents. In an embodiment, the solvents are acetone, for ruthenium nitrate or tetrahydrofuran.

Suitable ruthenium salts include, for example, ruthenium nitrosyl nitrate, ruthenium acetate, ruthenium carbonyl, ruthenium acetylacetonate, or the like. Other Ru^{+3, +4, +6, +7, and +8} known compounds may be used. In one embodiment, ruthenium acetylacetonate is used.

The calcined zeolite support is then impregnated in a dehydrated state with the solution of the metal salts. Thus, the calcined zeolite support should not be unduly exposed to atmospheric humidity so as to become rehydrated.

Any suitable impregnation technique can be employed including techniques well known to those skilled in the art so as to distend the catalytic metals in a uniform thin layer on the catalyst zeolite support. For example, the ruthenium along with the oxide promoter can be deposited on the zeolite support material by the “incipient wetness” technique. Such technique is well known and requires that the volume of solution be predetermined so as to provide the minimum volume which will just wet the entire surface of the zeolite support, with no excess liquid. Alternatively, the excess solution technique can be utilized if desired. If the excess solution technique is utilized, then the excess solvent present, e.g., acetone is merely removed by evaporation.

Next, the solution and zeolite support are stirred while evaporating the solvent at a temperature of from about 25° to about 85° C. until “dryness.”

The impregnated catalyst is slowly dried at a temperature of from about 110° to about 120° C. for a period of about 2 hours so as to spread the metals over the entire zeolite support. The drying step is conducted at a very slow rate in air.

The dried catalyst may be reduced directly in hydrogen or it may be calcined first. In the case of impregnation with ruthenium nitrate, direct reduction can yield a higher ruthenium metal dispersion and synthesis activity, but reduction of nitrates is difficult to control and calcination before reduction is safer for large scale preparations. Also, a single calcination step to decompose nitrates is simpler if multiple impregnations are needed to provide the desired metal loading. Reduction in hydrogen requires a prior purge with inert gas, a subsequent purge with inert gas and a passivation step in addition to the reduction itself, as described later as part of the ROR activation. However, impregnation of ruthenium carbonyl must be carried out in a dry, oxygen-free atmosphere and it must be decomposed directly, then passivated, if the benefits of its lower oxidation state are to be maintained.

The dried catalyst is calcined by heating slowly in flowing air, for example 10 cc/gram/minute, to a temperature in the range of from about 200° to about 350° C., for example, from about 250° to about 300° C., that is sufficient to decompose the metal salts and fix the metals. The aforesaid drying and calcination steps can be done separately or can be combined. However, calcination should be conducted by using a slow heating rate of, for example, 0.5° to about 3° C. per minute or from about 0.5° to about 1° C. per minute and the catalyst should be held at the maximum temperature for a period of about 1 to about 20 hours, for example, for about 2 hours.

The foregoing impregnation steps are repeated with additional substantially non-aqueous or aqueous solutions in order to obtain the desired metal loading. Ruthenium and other promoter metal and/or metal oxides are conveniently added together with ruthenium, but they may be added in other impregnation steps, separately or in combination, before, after, or between impregnations of ruthenium.

After the last impregnation sequence, the loaded catalyst zeolite support is then subjected to the ROR activation treatment of the present disclosure. The ROR activation treatment of the present disclosure must be conducted at a temperature considerably below 500° C. in order to achieve the desired increase in activity and selectivity of the ruthenium-impregnated catalyst. Temperatures of 500° C. or above reduce activity and liquid hydrocarbon selectivity of the ruthenium-impregnated catalyst. Suitable ROR activation temperatures are below 500° C., preferably below 450° C. and most preferably, at or below 400° C. Thus, ranges of 100° or 150° to 450° C., for example, 250° to 400° C. are suitable for the reduction steps. The oxidation step should be limited to 200° to 300° C. These activation steps are conducted while heating at a rate of from about 0.1° to about 5° C., for example, from about 0.1° to about 2° C.

The impregnated catalyst can be slowly reduced in the presence of hydrogen. If the catalyst has been calcined after each impregnation, to decompose nitrates or other salts, then the reduction may be performed in one step, after an inert gas purge, with heating in a single temperature ramp (e.g., 1° C./min.) to the maximum temperature and held at that temperature, from about 250° or 300° to about 450° C., for example, from about 350° to about 400° C., for a hold time of 6 to about 65 hours, for example, from about 16 to about 24 hours. Pure hydrogen is preferred in the first reduction step. If nitrates are still present, the reduction is best conducted in two steps wherein the first reduction heating step is carried out at a slow heating rate of no more than about 5° C. per minute, for example, from about 0.1° to about 1° C. per minute up to a maximum hold temperature of 200° to about 300° C., for example, 200° to about 250° C., for a hold time from about 6 to about 24 hours, for example, from about 16 to about 24 hours under ambient pressure conditions. In the second treating step of the first reduction, the catalyst can be heated at from about 0.5° to about 3° C. per minute, for example, from about 0.1° to about 1° C. per minute to a maximum hold temperature of from about 250° or 300° up to about 450° C., for example, from about 350° to about 400° C. for a hold time of 6 to about 65 hours, for example, from about 16 to about 24 hours. Although pure hydrogen is preferred for these reduction steps, a mixture of hydrogen and nitrogen can be utilized.

Thus, the reduction may involve the use of a mixture of hydrogen and nitrogen at 100° C. for about one hour; increasing the temperature 0.5° C. per minute until a temperature of 200° C.; holding that temperature for approximately 30 minutes; and then increasing the temperature 1° C. per minute until a temperature of 350° C. is reached and then continuing the reduction for approximately 16 hours. Reduction should be conducted slowly enough and the flow of the reducing gas maintained high enough to maintain the partial pressure of water in the offgas below 1%, so as to avoid excessive steaming of the exit end of the catalyst bed. Before and after all reductions, the catalyst must be purged in an inert gas such as nitrogen, argon or helium.

The reduced catalyst is passivated at ambient temperature (25°-35° C.) by flowing diluted air over the catalyst slowly enough so that a controlled exotherm of no larger than +50° C. passes through the catalyst bed. After passivation, the catalyst is heated slowly in diluted air to a temperature of from about 300° to about 350° C. (preferably 300° C.) in the same manner as previously described in connection with calcination of the catalyst.

The temperature of the exotherm during the oxidation step should be less than 100° C., and will be 50-60° C. if the flow rate and/or the oxygen concentration are dilute enough. If it is even less, the oxygen is so dilute that an excessively long time will be needed to accomplish the oxidation. There is a danger in exceeding 300° C. locally, since ruthenium oxides interact with alumina and silica at temperatures above 400° C. to make unreducible spinels, and above 500° C., Ru makes volatile, highly toxic oxides.

Next, the reoxidized catalyst is then slowly reduced again in the presence of hydrogen, in the same manner as previously described in connection with the initial reduction of the impregnated catalyst. This second reduction is much easier than the first. Since nitrates are no longer present, this reduction may be accomplished in a single temperature ramp and held, as described above for reduction of calcined catalysts.

The composite catalyst of the present disclosure has an average particle diameter of from about 1 to about 6 millimeters.

The charge stock used in the process of the present disclosure is a mixture of CO and hydrogen. The ratio of hydrogen to carbon monoxide is between about 0.5 and about 2.5, preferably between about 1 and about 2. Any suitable source of the CO and hydrogen can be used. The charge stock can be obtained, for example, by (i) the oxidation of coal or other forms of carbon with scrubbing or other forms of purification to yield the desired mixture of CO and H₂ or (ii) the reforming of natural gas. CO₂ is not a desirable component of the charge stocks for use in the process of the present disclosure, but it may be present as a diluent gas. Sulfur compounds in any form are deleterious to the life of the catalyst and should be removed from the CO—H₂ mixture and from any diluent gases.

The reaction temperature is suitably from about 160° to about 300° C., for example, from about 175° to about 280° C. or from about 185° to about 275° C. The total pressure is, for example, from about 1 to about 100 atmospheres, for example, from about 3 to about 35 atmospheres or from about 5 to about 30 atmospheres.

The gaseous hourly space velocity based upon the total amount of feed is less than 20,000 volumes of gas per volume of catalyst per hour, for example, from about 100 to about 5000 v/v/hour or from about 1000 to about 2500 v/v/hour. If desired, pure synthesis gas can be employed or, alternatively, an inert diluent, such as nitrogen, CO₂, methane, steam or the like can be added. The phrase “inert diluent” indicates that the diluent is non-reactive under the reaction conditions or is a normal reaction product.

The synthesis gas reaction using the catalysts of the present disclosure can occur in a fixed bed type of operation.

The following illustrative examples are intended to be non-limiting.

### EXAMPLES

### Example 1

### Preparation of 1% Ru/ZSM-5

Zeolite-supported ruthenium based catalyst (1% Ru/ZSM-5) was prepared by the impregnation method in a single step. A solution was prepared by dissolving ruthenium nitrosyl nitrate in water. This solution was added to the ZSM-5 zeolite (Si/Al=40) powder. After the mixture was stirred for 1 h at ambient temperature, the solvent was eliminated by rotavaporation. Then the catalyst was dried in an oven at 120° C. overnight and finally calcined at 300° C. for 2 h in a muffle furnace.

### Example 2

### Preparation of 1% Ru-0.5% Re/ZSM-5

Zeolite-supported ruthenium based catalyst (1% Ru-0.5% Re/ZSM-5) was prepared by the impregnation method in a single step. A solution was prepared by dissolving ruthenium nitrosyl nitrate and perrhenic acid in water. This solution was added to the ZSM-5 zeolite (Si/Al=40) powder. After the mixture was stirred for 1 h at ambient temperature, the solvent was eliminated by rotavaporation. Then the catalyst was dried in an oven at 120° C. overnight and finally calcined at 300° C. for 2 h in a muffle furnace.

### Example 3

### Preparation of 1% Ru-0.5% Re/Beta Zeolite

Zeolite-supported ruthenium based catalyst (1% Ru-0.5% Re/beta zeolite) was prepared by the impregnation method in a single step. A solution was prepared by dissolving ruthenium nitrosyl nitrate and perrhenic acid in water. This solution was added to the beta zeolite (Si/Al=19) powder. After the mixture was stirred for 1 h at ambient temperature, the solvent was eliminated by rotavaporation. Then the catalyst was dried in an oven at 120° C. overnight and finally calcined at 300° C. for 2 h in a muffle furnace.

### Example 4

### Preparation of 2% Ru/ZSM-5

Zeolite-supported ruthenium based catalyst (2% Ru/ZSM-5) was prepared by the impregnation method in a single step. Alumina (20% alumina) bound ZSM-5 zeolite (Si/Al=40) extrudate ( 1/16″ (1.6 mm)) was purchased from Zeolyst International. A solution was prepared by dissolving ruthenium nitrosyl nitrate in water. This solution was added to the zeolite extrudate. After the mixture was stirred for 1 h at ambient temperature, the solvent was eliminated by rotavaporation. Then the catalyst was dried in an oven at 120° C. overnight and finally calcined at 300° C. for 2 h in a muffle furnace.

Properties of the extrudate and catalysts of Examples 1-4 are set forth in Table 1.

TABLE 1

| | | | External | BET | |
|---|---|---|---|---|---|
| | | Micropore | Surface | Surface | |
| | Catalyst | Area, | Area, | Area, | Acidity |
| Examples | Composition | m²/g | m²/g | m²/g | μmol/g |
| 1 | 1% Ru/ZSM-5 | 256.7 | 164.5 | 421.3 | 280 |
| 2 | 1% Ru- | 252.4 | 164.5 | 416.9 | 290 |
| | 0.5% Re/ZSM-5 | | | | |
| 3 | 1% Ru- | 468.1 | 122.8 | 591.0 | 823 |
| | 0.5% Re/Beta | | | | |
| 4 | 2Ru/ZSM-5 | 194.3 | 173.0 | 367.3 | 317 |

### Example 5

### Activation of Metallic Catalysts of Examples 1-4

250 mg of Catalysts prepared as described in Example 1-4, diluted 50% by weight with gamma-alumina were each transferred to a 316-SS tube reactor having a 5 mm inner diameter. The reactor was then placed in a reactor furnace. The catalyst beds were flushed with a downward flow of argon for a period of two hours, after which time the gas feed was switched to pure hydrogen at a flow rate of 400 sccm. The temperature was slowly raised to 120° C. at a temperature interval of 1° C./minute, held constant for a period of one hour, then raised to 250° C. at a temperature interval of 1° C./minute and held constant for 10 hours. After this time, the catalyst beds were cooled to 180° C. while remaining under a flow of pure hydrogen gas. All flows were directed downward. The catalysts were contacted with synthesis gas at conditions set forth in Table 2.

### Example 6

### Fischer-Tropsch Activity

Catalysts prepared as described in Example 1-4 and activated as described in Example 5 were subjected to a synthesis run in which each catalyst was contacted with hydrogen and carbon monoxide in ratios between 1.5 and 2.0 at temperatures between 205° C. and 235° C., with a total pressure of 5-20 atm and a total gas flow rate of 2100-6000 cubic centimeters of gas (0° C., 1 atm) per gram of catalyst per hour. Results are set forth in Table 2.

TABLE 2

| | Example 1 | Example 2 | Example 3 | Example 4 |
|---|---|---|---|---|
| Composition | 1Ru/ | 1Ru-0.5Re/ | 1Ru-0.5Re/ | 2Ru/ |
| | ZSM-5 | ZSM-5 | Beta | ZSM-5 |
| T, ° C. | 215 | 215 | 215 | 215 |
| P, atm | 10 | 10 | 10 | 10 |
| SV, h⁻¹ | 2100 | 2100 | 2100 | 2100 |
| H₂/CO | 1.6 | 1.6 | 1.6 | 1.6 |
| CO Conv, % | 1.97 | 1.79 | 2.56 | 8 |
| C₁, wt % | 3.16 | 5.49 | 11.73 | 13 |
| C₂, wt % | 0.43 | 0.94 | 2.21 | 2 |
| C₃ + C₄, wt % | 7.04 | 7.64 | 13.2 | 27 |
| C₅ − C₁₂, wt % | 7.95 | 8.76 | 17.81 | 51 |
| C₁₂ − C₂₀, wt % | 81.42 | 77.17 | 55.05 | 7 |
| C₂₁₊, wt % | 0 | 0 | 0 | 0 |
| WTY_{Co} | 0.92 | 0.82 | 1.49 | 1.32 |
| gc h gCo | | | | |
| n-butane | 43.79 | 49.7 | 23.56 | 26.99 |
| Isobutene | 32.97 | 31.39 | 0.79 | 2.69 |
| 1-Butene | 1.36 | 0 | 10.25 | 7.15 |
| Isobutene | 13.93 | 11.96 | 5.74 | 19.35 |
| Cis-2-Butene | 2.94 | 2.62 | 22.56 | 16.86 |
| Trans-2-Butene | 5.01 | 4.33 | 37.1 | 26.96 |

The results for Example 4 illustrates the ability to substantially increase the yield in the gasoline range best with a reduction in the C₁₂-C₂₀ range and more C₄-formation

While various embodiments have been described, it is to be understood that variations and modifications may be resorted to as will be apparent to those skilled in the art. Such variations and modifications are to be considered within the purview and scope of the claims appended hereto.

What is claimed is:

1. A method for forming a catalyst for synthesis gas conversion, the method comprising:
impregnating a zeolite extrudate with ruthenium using a solution comprising a ruthenium salt to provide an impregnated zeolite extrudate; and
activating the impregnated zeolite extrudate by a reduction-oxidation-reduction cycle.

2. The method of claim 1, wherein the zeolite is selected from the group consisting of ZSM-5, ZSM-12, SSZ-32 and beta zeolite.

3. The method of claim 1, where in the ruthenium loading is from about 0.1 to about 15 wt %.

4. The method of claim 3, where in the ruthenium loading is from about 0.5 to about 5 wt %.

5. The method of claim 1, wherein the catalyst further comprises an element selected from the group consisting of Rh, Pt, Pd, Cu, Ag, Au, Zn, Cd, Hg, Mn, Pr and Re.

6. The method of claim 1, wherein the catalyst further comprises rhenium.

7. The method of claim 6, wherein the solution further comprises a rhenium salt.

8. The method of claim 1, wherein the ruthenium salt comprises ruthenium nitrate.

9. The method of claim 1, wherein impregnating the zeolite extrudate with ruthenium comprises multiple impregnations.

10. The method of claim 9, wherein the multiple impregnations further comprise intervening drying and calcination treatments and, wherein the calcination treatments comprise calcination in air.

11. The method of claim 1, wherein the reduction-oxidation-reduction cycle is conducted at a temperature in a range of about 100° to about 450° C.

12. The method of claim 11, wherein the reduction-oxidation-reduction cycle is conducted at a temperature in a range of about 250° to about 400° C.

13. The method of claim 12, wherein:
a first reduction step of the reduction-oxidation-reduction cycle is conducted at a temperature in a range of about 200° to about 450° C.;
an oxidation step of the reduction-oxidation-reduction cycle is conducted at a temperature in a range of about 250° to about 350° C.; and
a second reduction step of the reduction-oxidation-reduction cycle is conducted at a temperature in a range of about 200° to about 450° C.

14. The method of claim 1, wherein the activating is conducted while heating at a rate of from about 0.1° to about 2° C. per minute and wherein a first reduction step of the reduction-oxidation-reduction cycle is conducted in the presence of substantially pure hydrogen.

15. A hybrid Fischer-Tropsch catalyst comprising a zeolite support impregnated with ruthenium and wherein the support is selected from the group consisting of ZSM-5, ZSM-12, SSZ-32 and beta zeolite.

16. The catalyst of claim 15, wherein the catalyst further comprises an element selected from the group consisting of Re, Rh, Pt, Pd, Ag, Au, Mn, Zn, Cd, Hg, Cu, Pr or other rare earth metals.

17. The catalyst of claim 15, wherein the zeolite support impregnated with ruthenium further comprises rhenium.

18. A method of performing a synthesis gas conversion reaction, the method comprising contacting the catalyst of claim 15 with synthesis gas at a reaction temperature of 160° C. to 300° C., and H₂ to CO ratio of 1.5 to 2.0 a total pressure of 3 to 35 atmospheres, and an hourly space velocity 100 to 5000 v/v/hour.

19. The method of claim 18, wherein products of the synthesis gas conversion reaction over a catalyst having ruthenium 0.8 to 1.2 weight % including from 0.0 to 0.7 weight % rhenium on a support selected from the group consisting of ZSM-5, ZSM-12, SSZ-32 and beta zeolite and wherein the products comprise 1-15 weight % CH₄; 4-15 weight % C₂-C₄; 70-90 weight % C₅₊; and less than 1 weight % C₂₁₊.

20. The method of claim 18, wherein products of the synthesis gas conversion reaction over a catalyst having 1.5 to 2.5 weight % ruthenium on a ZSM-5 support and wherein the products comprise 10-15 weight % CH₄; 25-35 weight % C₂-C₄; 70-90 weight % C₅₊, wherein 40-50 weight % is in the C₅-C₁₂; and less than 1 weight % C₂₁₊.
